(19) United States
(12) Patent Application Publication
(10) Pub. No.: US 2024/0108380 A1
(43) Pub. Date: Apr. 4, 2024

(54) Fixture For Securing And Aiding Frontal Placement Of Surgical Equipment To A Bore In Skull Bone

(71) Applicant: InTRAvent Medical Partners, L.P., Hummelstown, PA (US)

(72) Inventors: Adam J. Barner, Hershey, PA (US); Nick Oblas, Philadelphia, PA (US); Tom O'donnell, Philadelphia, PA (US); Gregory James Maino, Chester Springs, PA (US); Jonathan Slocum, Litchfield, NH (US)

(73) Assignee: InTRAvent Medical Partners, L.P., Hummelstown, PA (US)

(21) Appl. No.: 18/200,858

(22) Filed: May 23, 2023

Related U.S. Application Data
(60) Provisional application No. 63/413,026, filed on Oct. 4, 2022.

(51) Int. Cl. A61B 17/68

(52) U.S. Cl. CPC A61B 17/68; A61B 2017/681

(57) ABSTRACT

Disclosed is a surgical fixation device to mount on bone. This device does not utilize screws and achieves stability via applying force to the inner wall of the bore and is therefore faster, easier to use, and less user dependent. The device facilitates fast, easy anchoring system for use with other devices that require rigid fixation to bone. The device provides for intermittent mounting of tools, equipment, and/or accessories to a cylindrical bore in bone for use in surgical applications without compromising surrounding bine structure such as done by the use of bone screws. Rigid fixation to bone is a common requirement for surgical procedures for placement of tools, monitoring systems, positional systems, or used as fiducial markers. The present fixation device removably mounts on bone without the disadvantages associated with bone screws and other fixation devices.

### CROSS REFERENCE TO RELATED APPLICATION

This application claims priority from U.S. provisional application No. 63/413,026 filed Oct. 4, 2022, the disclosure of which is hereby incorporated herein by reference.

### TECHNICAL FIELD

The present disclosure relates to an intermittent mounting device that may be disposed within bone or about a bore in a skull and used in surgical procedures and applications thereof.

The present disclosure also relates to a removable mounting device for rigid fixation of surgical instruments, tools and/or accessories for purposes of registration, guidance, and monitoring without the use of bone screws for the rigid fixation.

### BACKGROUND

The emergence of modern neurosurgical procedures as well as other surgical and non-surgical procedures has created an increased need to mount devices on the patient's bone structure such as the skull and other bone structures. Different biomedical devices and surgical instruments have been developed to better serve the medical practice that utilize and need such a mounting device. Most current technology utilize bone screws and the like. However, these types of fixation devices have many drawbacks including, in some cases, causing trauma to the nearby bore sites and potential weakening of the surrounding bone structure where the bore is located.

Although bone screws are functional and in many cases state of the art, medical practitioners continue to strive for a more optimal device to provide rigid fixation and ease of removal and insertion for intermittent use. Some of the difficulties encountered with state of the art bone screws and other similar fixation devices include, for example, the difficulty of managing such small screws by the medical professional or difficulty in removing the device in case replacement is needed.

Indeed, if the device needs to be quickly removed, the medical practitioner must unscrew all the inserted screws in the bone structure. If the device needs to be remounted the bone may be compromised by the previous holes causes by the prior screw insertions. A better device that meets improved functionality criteria is still needed in the medical profession.

Numerous attempts have been made to overcome problems with screw fixation in compromised bone quality. These include modified designs such as conical screws, special thread geometries, expandable screws, cross-linked constructs, and screw coatings, and alternative materials for screws such as composite or polymer based screws. Alternatively, surgical techniques have been modified to avoid unwanted consequence even though this is not commonly done. However these prior attempts have still failed in the case of osteoporosis where the surface of the bone is brittle and bone fragility and fracture risk are increased.

Therefore, an alternative securing device has great importance. There is a need for an improved device that may mount over a bone structure and void the above disadvantages.

### SUMMARY

Compared to the above prior attempts, the present disclosure fulfills the above criteria and provides additional benefits that state of the art systems cannot provide. Disclosed and described is a fixture device that secures inside, around, or underneath a bone bore. Once the initial hole is made in the bone for a medical procedure, no screws are needed to secure the fixture device over the bone.

The fixture device may be used to hold surgical instruments, imaging devices, tools, and other accessories depending on the procedure. Additionally, the rigid fixation to bone allows for instant registration to patient anatomy for use with imaging devices, compared to other modern devices that require complex and lengthy calibration processes. The novel holding mechanism to hold onto the inside of a bore, for example in a skull or bone, may be easily and quickly secured and removed.

The novel holding mechanism allows intermittent holding or removal and then subsequent reuse of the device without compromising the securing bone structure by screwing and unscrewing in bone structure every time the device is placed again on the bone structure.

Similar devices are commonly and almost exclusively fastened to a bore or hole through the uses of bone screws in the outer table. Insertion and removal of small bone screws by hand can be tedious and time consuming, particularly for applications where the device is not for use as an implant and only intended to be used intraoperatively. The present invention and device does not utilize screws. The present device achieves stability via applying force to the inner wall of the bone bore and is therefore faster, easier to use, and less user dependent than state of the art bone fixation devices used to hold accessories and the like. The present device facilitates fast, easy anchoring system for use with other devices, accessories, monitors, diagnostic equipment, and the like that require rigid fixation to bone.

The device for intermittent mounting of tools, equipment, and/or accessories to a cylindrical bore in bone for use in surgical applications has several embodiments as further discussed herein. Rigid fixation to bone is a common requirement for surgical procedures for placement of tools, monitoring systems, positional systems, or used as fiducial markers and the present invention accomplishes this fixation without any of the drawbacks of screws or other prior attempts to provide a rigid fixation to bone.

In some implementations, the anchoring device has a flexible structure which may be deformed by a user to then apply a set preload force onto a bone bore, either inside the bore or on one of the surfaces in order to hold the device in place with friction

In some implementations, the flexible structure is an hour-glass shaped flexure which when deformed, allows the legs of the anchoring device to fit inside a bone bore, and then when released, the flexure remains deformed but applies a force to the inside of the bone bore.

In some implementations, the anchoring device deforms via a hinge and is actuated via a screw to hold it in desired positions.

In some implementations, the anchoring device has raised features on surfaces meant to contact bone, to help give the device more bite into the bone.

### BRIEF DESCRIPTION OF THE DRAWINGS

To assist those of skill in the art in making and using the disclosed composition and method, reference is made to the accompanying figures, wherein:

FIGS. 1A-1B illustrate one embodiment of the present invention that utilizes a dove tail threaded slide and sliding tooth to mount the device inside a bone bore;

FIGS. 2A-2B show an illustrative design of one embodiment of the present invention that utilizes a retractor mechanism and four feet or protrusions to mount the device inside a bone bore;

FIG. 3A-3B show an illustrative design of one embodiment of the present invention that utilizes a set screw mechanism and two feet or protrusions to mount the device inside a bone bore;

FIG. 4 shows an illustrative design of one embodiment of the present invention that utilizes a notch mechanism and two feet or protrusions to mount the device inside a bone bore; and

FIGS. 5A-5B illustrate additional views of the ‘dovetail concept described in 1A-1B;

FIG. 6 a side view of the device depicted in FIG. 1A;

FIG. 7 depicts the deforming hinge (120) that flexibly joins a first tooth body (110) and a second tooth body (113);

FIG. 8 depicts a screw and cantilever embodiment that utilizes the principles in the above drawings;

FIG. 9 depicts a side screw cantilever device utilizing the principles in the above drawings;

FIG. 10 depicts the deforming hinge (120) that flexibly joins a first tooth body (110) and a second tooth body (113) as shown and described in FIG. 7;

FIGS. 11A, 11B, 11C depict revolved teeth features intended to apply force into the wall of the bone bore for use in any of the embodiments described herein;

FIG. 12 depicts one of the embodiments with intermittent mounting of tools, equipment, and/or accessories to a cylindrical bore in bone for use in surgical applications;

FIG. 13 depicts one embodiment showing devices or features intended to guide other devices, such as but not limited to, catheters, biopsy needles, stents, endoscopes, ultrasonic probes, DBS leads or other intraoperative devices) in, therethrough, or around bone or other anatomy;

FIG. 14 depicts one embodiment for placement of temporary or permanent monitoring, measuring or diagnostic devices in, therethrough, or around bone or other anatomy;

FIG. 15 depicts the embodiment of FIG. 14 showing use of the assembly with other anatomy with a mounting feature and accessory; and

FIG. 16 depicts a cross section of FIG. 15 showing the lock screw moving the revolved teeth features.

### DETAILED DESCRIPTION

The invention includes, according to certain embodiments, systems and processes relates to a fixation device used in medical procedures.

Disclosed is a device for intermittent mounting of tools, equipment, and/or accessories to a cylindrical bore in bone for use in surgical applications. Rigid fixation to bone is a common requirement for surgical procedures for placement of tools, monitoring systems, positional systems, or used as fiducial markers.

Similar devices are commonly and almost exclusively fastened to a bore or hole through the uses of bone screws in the outer table. Insertion and removal of small bone screws by hand can be tedious and time consuming, particularly for applications where the device is not for use as an implant and only intended to be used intraoperatively. This device does not utilize screws and achieves stability via applying force to the inner wall of the bore and is therefore faster, easier to use, and less user dependent. The device facilitates a fast, easy anchoring system for use with other devices that require rigid fixation to bone.

A device for intermittent mounting of tools, equipment, and/or accessories to a cylindrical bore in bone for use in surgical applications. Rigid fixation to bone is a common requirement for surgical procedures for placement of tools, monitoring systems, positional systems, or used as fiducial markers.

Similar devices are commonly and almost exclusively fastened to a bore or hole through the uses of bone screws in the outer table. Insertion and removal of small bone screws by hand can be tedious and time consuming, particularly for applications where the device is not for use as an implant and only intended to be used intraoperatively. This device does not utilize screws and achieves stability via applying force to the inner wall of the bore and is therefore faster, easier to use, and less user dependent. The device facilitates fast, easy anchoring system for use with other devices that require rigid fixation to bone.

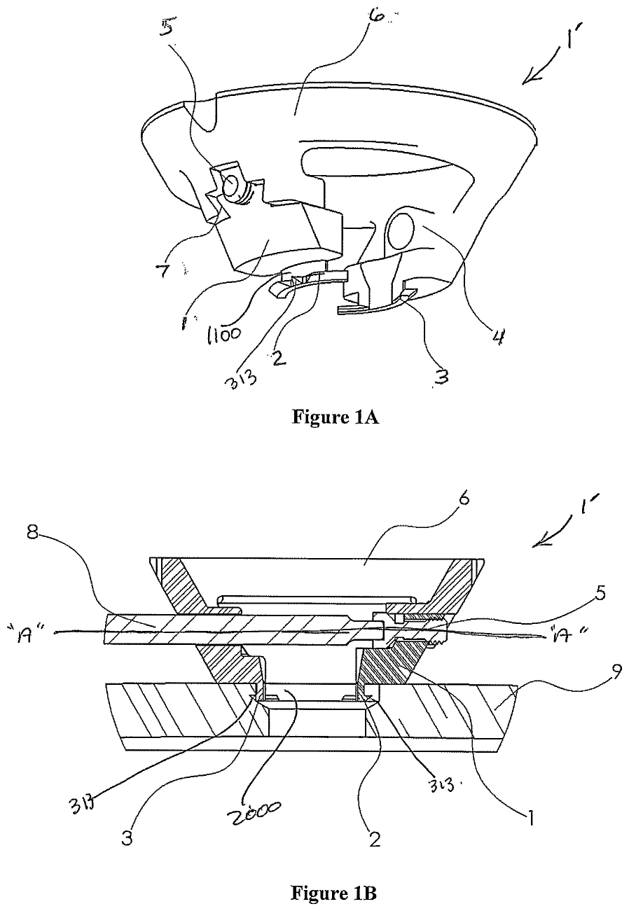

FIGS. 1A-1B show an isometric and cross section of an embodiment of a so-called two part or multiple part rigid anchoring device referred to also as a “dovetail concept.” The device (1′) may contain a dovetail feature wherein the male feature can contain a sliding tooth (2) and the female feature (7) is part of a separate different body that contains a fixed tooth (3). Female feature (7) is a recess in the device that also houses a moving detail base (1). The moving dovetail base (1) feature contains sliding tooth (2) and moves in a traverse or horizontal direction. Moving dove tail base (1) of this dove tail feature contains a thread for actuation via screw (5). Driving guide (4) is a through hole to position a driver (8) on the same axis “A” as screw (5).

Screw (5) may be a left handed screw such as a counterclockwise rotation (CCW) to provide a user with consistent right handed tightening nomenclature. Turing the driver (8) via screw (5) to the right or clockwise (CW) applies force to a bore hole or an opening (2000) in a bone structure (9). An annular body (6) allows for versatile mounting on any bone structure of various accessories and tools. Such accessories and tools may be for surgical use, monitoring, evaluation, and the like. Again, the female dove tail feature or recess (7) retains the male dove tail feature or moving dove tail base (1). Recess (7) allows for screw (5) to be aligned with the direction of the dove tail cut, and the axis “A.”

Therefore, tooth (2) moves in a horizontal direction relative to tooth (3) and tooth (3) is stationary. Depending on the embodiment, features with the tooth may include a sharp (313) and an extender (1100) connected to the sharp and tooth. Because of this screw-less design, less trauma is given to the surrounding bone structure in a skull bore or bore in any other bone. The present anchoring device is intermittently removed easier than the state of the art screw anchoring device because only one lock thread (5) is required to anchor the device as compared to multiple screws required in the state of the art device to anchor the state of the art device. These multiple screws are also miniature making it much more difficult to manage as compared to the single and larger lock thread (5) in the present invention.

Within the moving dovetail (1), a left-hand screw or a lock thread (5) is aligned axially with the cut direction in a bone structure. The dovetail (1) contains the screw (5) that when turned clockwise, for example, moves with driver (8). Driver (8) may or may not be threaded like screw (5) depending on the embodiment. The moving dovetail base (1) because of the dove tail extends away in a horizontal direction from the fixed foot containing stationary tooth (3). Sliding tooth (2) and fixed tooth (3) apply equal and opposite force to the inner wall of a bore. Turning the screw (5) counterclockwise reverse the direction of the sliding foot (2) to release the device from bone.

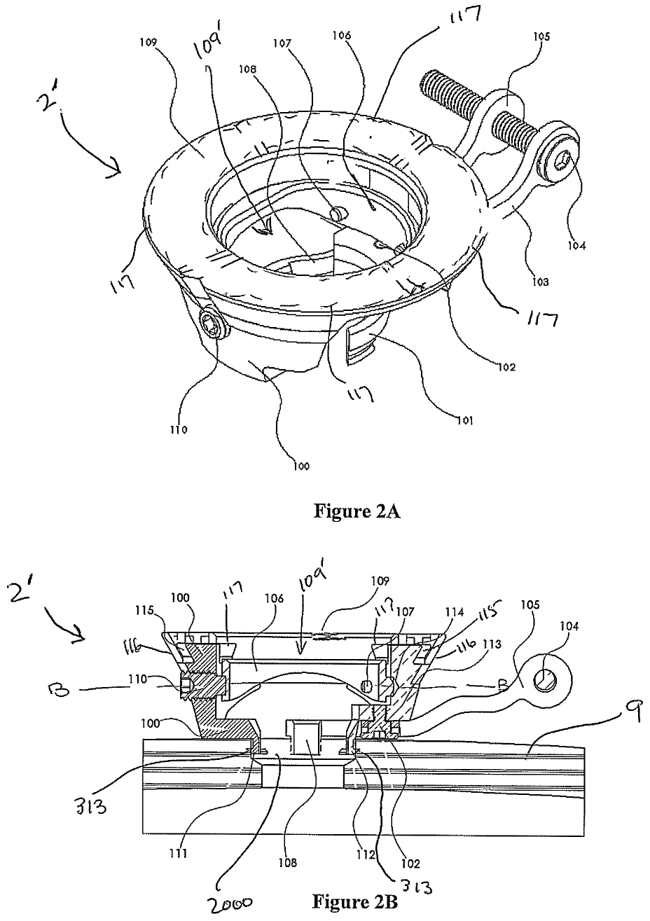

FIGS. 2A-2B show an isometric and cross sectional view, respectively, of an embodiment of a multipart retractor style anchoring device (2′). A body containing a tooth feature or base or tooth body A (100) and a separate body containing a different tooth feature or different base or tooth body B (113) are positioned opposite each other. A set screw (110) is connected to base (100). The set screw (110) controls movement of base (100) in a horizontal track such that sharps (313) and tooth (111) and tooth (112) move in opposite directions and are secured in a bore bone opening (2000).

The set screw (110) when tightened applies force on a spreader ring (106). The set screw (110) pushes ring (106) and applies force on ring (106) in an opposite direction of tooth body A or base (110) for applying opposite directional forces to each body (100) and (113). Depending on the embodiment, each body tooth body A (100) and tooth body B (113) are connected via a non-plastically deforming hinge member (117) is shown by hidden lines may be disposed below annular body (109). Hinge member (117) allows for expansion and contraction of bodies (100) and (113) as set screw (110) applies force to spreader ring (106). An alignment pin (107) or a plurality of alignment pins (107) are used to keep the spreader ring (106) aligned in the direction of force being applied to the ring by the screw (110). An annular body (109) defines a window (109′) that allows 360 degree access inside the bore hole (2000).

A tension screw (104) tightens and opens retractor teeth A (101) and retractor teeth B (108) to apply side forces to the bore (2000) in relationship to the front and back forces applied by set screw (110) to ring (106) and hence applying forces to body A (100) and body B (113) on the bore hole, respectively. At least one tooth A (111) is disposed on tooth body A (100). And at least one a tooth B (112) is disposed on tooth body B. Both tooth members (111) and (112) are in contact with the walls of the bore hole (2000) of bone structure (9) to provide intermittent support to the device such that the device may be easily removed with any accessories or other attachments on the device without the need of removing any bone screws that are attached to bone structure (9). This feature allows easy removal and then reattachment to bone structure (9) while minimizing any trauma to bone structure (9) or weakening the bone structure that surrounds bore (2000).

The tension screw (104) is contained by retractor A leg member (105) and retractor B leg member (103) that both move simultaneously teeth A (101) and teeth B (108) in a widening and constricting manner depending on the turning direction of tension screw (104).

A retractor pivot pin or shoulder pin (102) is disposed adjacent to and inside tooth body B (113). The pivot pin allows leg members (103) and (105) and hence retractor tooth member A (101) and retractor tooth member B (108) to pivot in relationship to each other in the same plane. Body (113) may contain a thread to receive a shoulder screw, pin, or cylindrical constraining feature (102). The pin (102) again may function as a pivot point for the pair of retractor members (105), (103). Retractor member (103) may contain mating features (104) for use with a fastener, ratcheting feature, or mechanical locking feature to bring distal ends of (103) and (105) towards each other. As distal ends of the retractor members are tightened, retractor teeth (101) and retractor A tooth member (108), plurality of teeth also known as feet, wherein each teeth are also known as a foot, expand into the wall of the bone bore. The force applied by retractor teeth (101) and (108) can be perpendicular that of tooth (111) and tooth (112). Each tooth may have a sharp (313), a sharp edge, a plurality of teeth or any combination thereof depending on the embodiment.

Further shown in FIG. 2B is a feature of a ring force application dipple (114). Dipple (114) is provided to focus force on a side wall of tooth body B (113). An angle under-cut groove (115) allows tooth bodies A (100) and tooth body B (113) to be constrained along a direction of force in “axis B” applied by the ring (106). Groove (115) also provides rigidity to the annular body (109) such that when the system is under tension, for example when ring force is applied and the teeth (111) and teeth (112) apply pressure to the bone structure (9), all components of device (2′) are fully constrained to provide rigid fixation. Also include in the device, depending on the embodiment is a stabilizing feature (116) for either or both tooth body A (100) and/or tooth body B (113). Stabilizing feature 116 may, for example, mate into groove (115) when the device (2′) is under tension and the teeth (111) and teeth (112) are spread apart and/or retractor teeth (108) and retractor teeth (101) are spread apart.

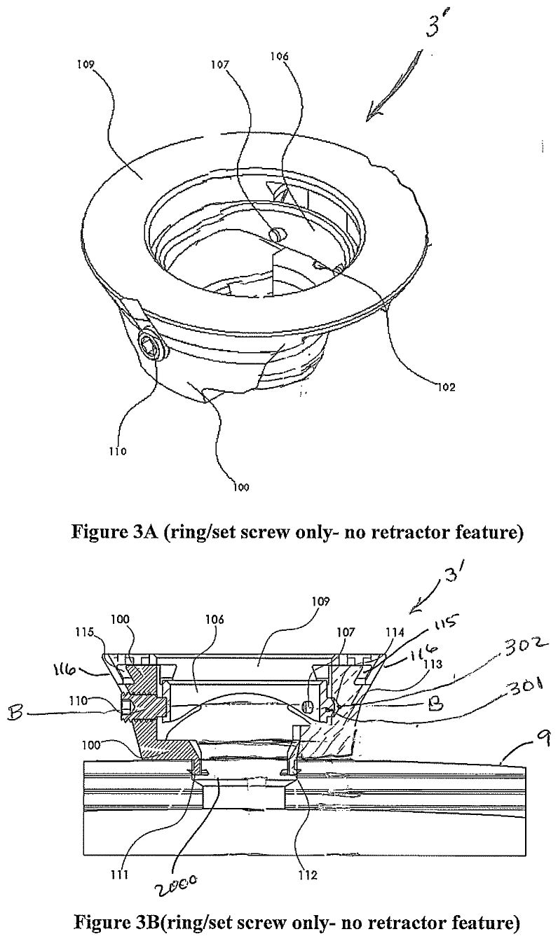

FIGS. 3A-3B illustrate a concept for device (3′). Functions, structures, and reference numbers used above for devices (1′) and (2′) apply to device (3′). In device (3′) the retractor features (103) and (105) are removed. Set screw 110 is utilized to move body A (100) and body b (113) through force applied by the set screw to the ring (106). Shown in FIG. 2B the alignment pin (107) allows the force applied to ring (106) to be guided in a direction along axis B. The ring (106) may also contain a detent (301) and matching detent recess (302) to assist in mechanical movement of body B (113) and/or body A (100).

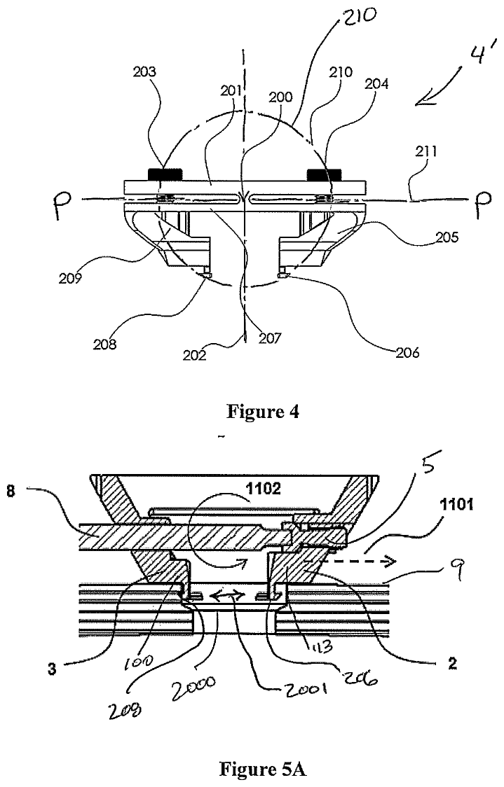

FIG. 4 shows an embodiment of a single part anchoring device (4′) wherein a mechanical notch (200) can be used to apply predictable strain to the body material such that tooth (208) and (206), plurality of tooth also known as feet and each tooth also known as foot, expand and/or contract in opposite directions, tangent to the drawn circular path or rotation line or teeth expansion path (210). A ratio was determined of thickness of higher member (201) and lower member (207) to maximize force applied by tooth B (208) and tooth A (206) while maintaining parallelism of the top surface of the annular portion of (201). It was found that the thickness of member (201) when thicker than member (207) surprisingly gave more maximum force than if the members were of equal thickness or if thicknesses were reversed (for example if member (207) was thicker than member (201). Vertical axis (202) illustrates an axis that is parallel to the bore hole (2000) in bone (9) shown in the figures. Depending on the implementation the bore hole may be approximately 14 mm, 11 mm, and 8 mm in diameter. However, the devices described herein, and the principles of the invention are not limited to any specific diameter of a bore hole and may be utilized with any drill hole size or shape and size of any opening in any bone structure.

A pair of tensioning screw A (203), and tension screw B (204) can be turned clockwise and/or counterclockwise to apply deformation about the notch, where a deforming body A (205) and a deforming body B (209) move towards or away from plane P (211) along rotation line (210), depending on the tightening or loosening of tension screws (203) and (204). Deforming body A and deforming body B may be deformed in opposite direction or the same direction along path (210) depending on loosening or tightening of their respective tension screw. Thickness ratios, for example, include but are not limited to a range for higher member to lower member, respectively, from 10:1 through 2:1, and most preferably 3:1.

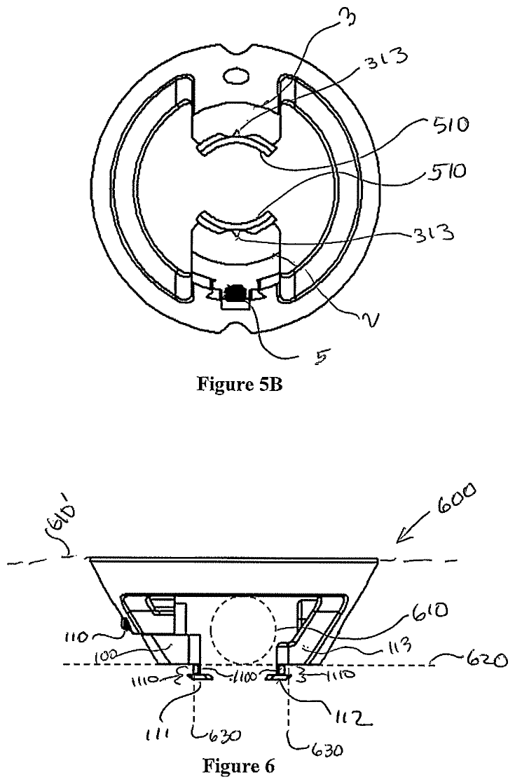

FIG. 5A-5B are additional views of the ‘dovetail concept described in FIGS. 1A-1B. FIG. 5A is a cross section view of the device shown in FIG. 1A through the midline. In one embodiment, as driver (8) is rotated in the clockwise direction (1102), the left-handed thread shoulder screw (5) engages the sliding tooth body (2) and forces it to travel in a direction (1101) away from the fixed tooth (3) within bone bore hole (2000) and tangent to the mounting surface of bone structure (9). This embodiment provides a spring action or movement in the direction of directional lines (2001) for teeth (208) and (206) where there is material deflection in the device without deformation to apply pressure to the inner wall of the burr hole or bone hole (2000). The device is removable and/or temporary and removal does not cause relevant damage to the bone structure (9) as compared to current state of the art device that are attached to the bone structure.

FIG. 5B depicts a top view of the device in FIG. 5A fully expanded. Depending on the embodiment the tooth bodies (2) and (3) may also contain, each or individually, a sharp (313) and/or a sharp edge (510) to contact the walls of the bone hole (2000). Again it is within the scope of this invention to have multiple rows of teeth and/or multiple rows of sharps and/or sharp edges. The teeth may, depending on the embodiment, be a rough feature. Such a rough feature may include, but is not limited to surface protrusions, sharp edges, rough edges, sharps or sharps in a triangular shape or pyramidal shape or any other geometrical shape, surface texture, surface finish, any deviations in the direction of the normal vector of the real surface of the device in contact with the wall of the bone bore from its ideal form, and the like. The teeth and/or protrusions are intended to bite into and engage the sidewall of the burr bone hole as the device's teeth is expanded. Teeth can be of any profile and size. Depending on the embodiment teeth could also be a knurl, roughened surface, or tapered protrusion depending on the expected loading (pullout, rotation, torque) In addition, the arc length within the burr hole is considered for the necessary interactions of items that need to go into or through the burr hole. It is possible to have multiple rows of teeth depending on the clinical need. The clearance through the center of the device allows for access through burr hole as clinically necessary. In addition, lateral access is available through the space from the side of the device to visualize and access anything such as, but not limited to, implants, instruments, catheters, and the like, which may be going into the burr hole.

FIG. 6 is a side view of the device depicted in FIG. 1A. The device is such that the bore in the bone is accessible from either side of the teeth (111), (112) engaged inside the bore and under a mounting surface or bone structure (9). This is critical for intermittent access and visibility of instruments used within the bore without impeding fixation of registration, guidance, instruments, and/or monitoring devices and the like. The device, depending on the embodiment, may or may not have a surface that does not deform and/or deflect, and is treated as a reference surface for interface and/or mounting with other components as previously described. The device sits flat on circular surfaces and thus is flat (locally tangent) to the circular surface of the bone or skull because the anchoring of the device is within the bone hole or bone burr instead of anchoring the device on the surface of the bone or skull as done in state of the art devices. This feature provided by the present device is an advantage over prior art devices. The present device provides such a flat surface for mounting any accessory on any geometrically shaped bone structure due to the anchoring mechanism being in the bone burr instead of the surface of the bone.

Also shown in FIG. 6 is a set screw (110) that has similar properties and functions as the previous set screws described herein. Set screw (110) moves at least one of a revolved teeth or foot (1110) along axis line (620) that represents the surface of bone structure (9). Depending on the embodiment, foot (1110) may have an extender (1100) that extends vertically inside a bone bore alone axis line (630) to allow teeth (111) and (112) to attach deeply within the bone burr or bone hole of the bone structure. As shown in halo (610) access to the bore hole from the top of device (600) as well as the side between teeth (111) and (112) allows the user a full 360 degree access to the bore hole at least in the planes represented by (610) and (610′).

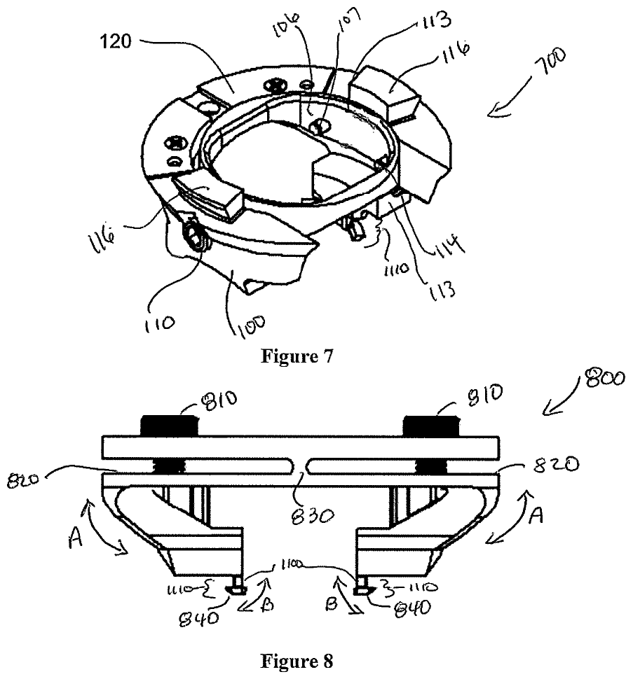

FIG. 7 depicts the deforming hinge (120) that flexibly joins a first tooth body (100) and a second tooth body (113) of device (700). A set screw (110) can be turned clockwise to apply force onto an annular body (106), which in turn applies force to the opposing tooth body (113). Depending on the embodiment, device (700) may have the stabilizing feature (116) for the tooth bodies or one tooth body as described in FIGS. 2A-2B. This stabilizing feature mates into angle under-cut groove (115) previously shown and described in FIG. 2B. Annular body (106) is alignment can be maintained by alignment pins (107) and (114). Again the bodies (110) and (113) either individually or in combination may contain the stabilizing feature (116) that has the same structure and function that mates into cavity or angle undercut groove (115) of the same geometry in annular body (109). The stabilizing features (116) mating with grooves (115) constrains body (100) and (113) such that the force applied through the set screw (110) can be efficiently transferred to teeth in the burr hole (111), (112). It also provides rigidity to the annular body such that when the system is under tension (ring force applied, teeth apply pressure to bone), all components are fully constrained to provide rigid fixation. This stabilizing and rigid fixation effects are the same as provided and described in the device shown in that of FIGS. 2A-2B.

FIG. 8 depicts a screw and cantilever device (800) that utilizes the principles of the invention. Shown are tightening screws (810) that when tightened and/or loosened contacts surface (820) bending the surfaces like a cantilever about intersection (830) in an upward or downward directional movement as shown in arrow (A). This upward or downward directional movement (A) then moves teeth (840) in a left or right directional movement as shown in arrows (B). Again revolved teeth (1110) is inserted inside the bone burr or bone hole to secure the devices of the present invention and may include extenders (1100) to reach the teeth (840) deeper into the bone burr or bone hole.

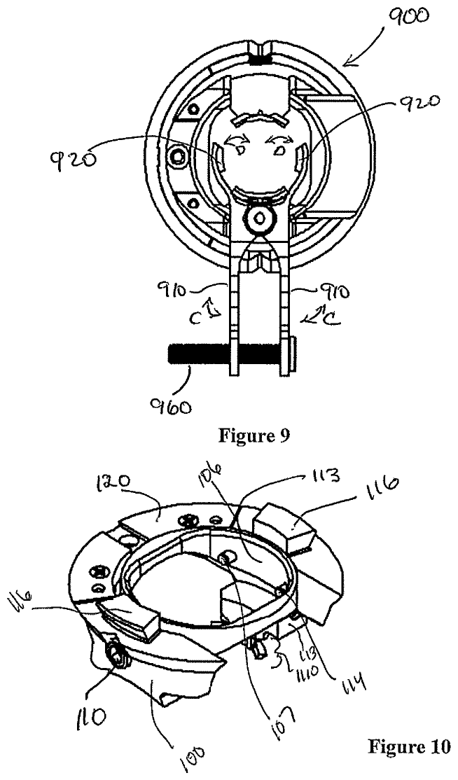

FIG. 9 depicts a side screw cantilever device (900). Shown is side screw (960) that may be tighten or loosen. The tighten and loosening move a plurality of legs (910) in a generally rightward and leftward motion shown on the arrows C. This generally rightward and leftward motion moves teeth (920) in a motion shown on arrows D.

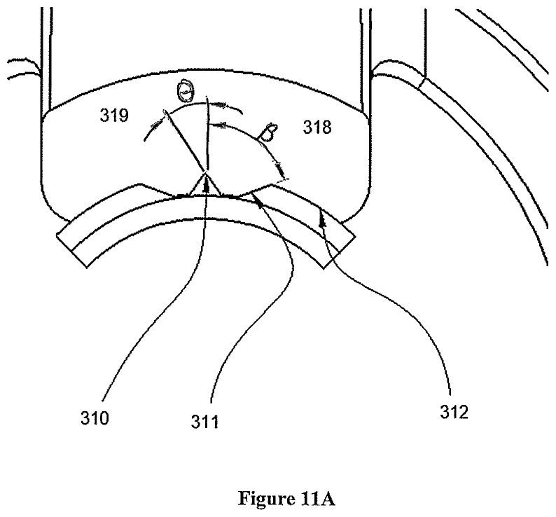

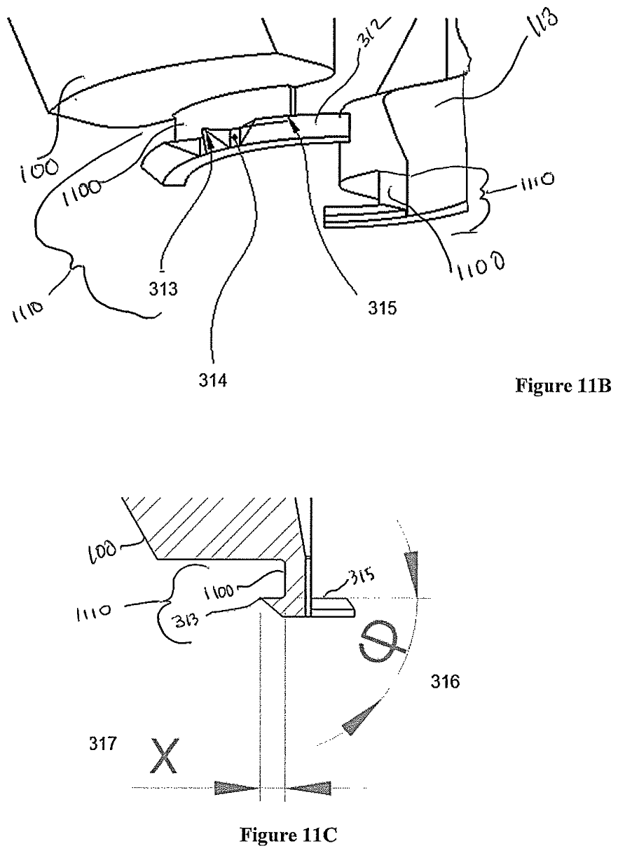

FIG. 10 depicts the deforming hinge (120) has the same function and structure previously described as for deforming hinge (117) that flexibly joins a first tooth body (100) and a second tooth body (113) as shown and described in FIG. 7. Wherein similar reference numbers identify similar elements having the same structure and function previously described herein. It is being reproduced herein and shown as merely an exemplary embodiment for the teeth features further shown and described in FIGS. 11A, 11B, and 11C.

FIGS. 11A, 11B, and 11C depicts shows revolved teeth features intended to apply force into the wall of the bone bore for use in any of the embodiments described herein.

FIG. 11A, 11B, 11C shows revolved teeth or foot (1110) features intended to apply force into the wall of the bone bore for use in any of the embodiments described. Each foot (1110) may have one or more features of the following depending upon the embodiment. An extender (1100) is disposed from base (100) and may or may not be part of the revolved tooth (1110). A sharp (310) can contact the bore wall first and penetrate to prevent rotation about the bore axis. The sharp (310) may, depending on the embodiment, have a triangular shape with a sharp point. Sharp (310) may also be shaped in a circular, or jagged tooth geometry, which would contain more than one sharp point, or any other geometrical shape including but not limited to cylindrical, circular, oval, polygonal and the like. In addition the sharp may or may not include a rough surface, or texture and/or be replaced with a rough surface or texture and the like as previously described. Leading edge's angle (319) 0 in FIG. 11A, includes but is not limited to, an angle between 0 and 90 degrees and a trailing edge angle (318) B has an angle between 0 and 120 degrees. A trailing edge (311) may or may not also contain a sharp edge for additional security. The present inventors found that both the leading edge and trailing edge angles have a synergistic effect to the function of the sharp (310) that allows initial secure holding into the bone structure without generating significant bone damage as compared to that of typical state of the art bone screw.

A revolved sharp surface (312), depending on the embodiment, may be concentric as shown in FIG. 11A with the bore or follow a circular shape where the circle lies on the same line or axis as the center of the bore. The revolved sharp surface may contain any of the surface roughness, surface textures, teeth, sharps, geometrical shapes, edges, and the like described. A spacer (314) may separate the sharp point (313) and the revolved sharp surface (312) in any of the embodiments also described. As shown in FIG. 11B, tooth body A (100) and tooth body B (113) may contain one or more revolved sharp edge (315) resembling a knife edge, for example, that is parallel with any other teeth in any other combination of teeth described. A cross section of the revolved sharp (315) is shown in FIG. 11C and defined by tooth length distance (317) and tooth angle (316) between 1 and 80 degrees. Depending on the embodiment the tooth length distance (317) may include a distance of about 0.0001 mm to about 100.0 mm, and any other distance that may be needed for the particular application of the device.

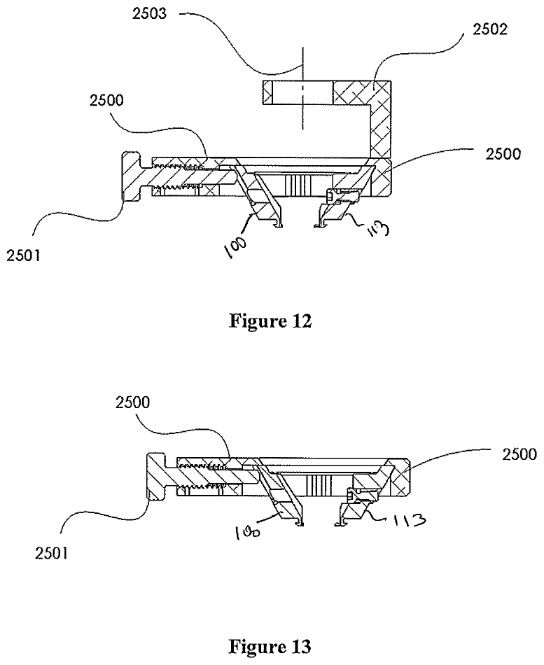

FIG. 12 depicts one of the embodiments with intermittent or temporary mounting of tools, equipment, and/or accessories to a cylindrical bore in bone for use in surgical applications, and the like. Indeed, in a prior art device if the prior art device and the related mounting and accessories and the like attached to the prior art device needed to be quickly removed, the medical practitioner must unscrew all the inserted screws that are in the bone structure. If the prior art device needs to be remounted, the bone may be compromised by the previous holes causes by the prior screw insertions. The present invention's principles provide for a device of that meets improved functionality criteria, namely intermittent or temporary mounting of tools, equipment, and/or accessories into a cylindrical bore in the bone. Unlike state of the art device that mount on the bone structure, the present invention mounts within the bone burr or hole itself providing for less trauma if the device and related tools, equipment, and/or accessories and the like need to quickly be removed and then put back in place.

Shown in FIG. 12 is one embodiment of the present bone fixation device with various accessories. It is understood that the present invention's bone fixation devices cover use with any and various accessories used in the medical field. The following is shown merely as an example and the fixation device is not limited to a specific mounting device and/or any accessories and can be used with any mounting device and/or any accessories. As an example, mounting device (2500) seats to the bone fixation devices utilizing the concepts described. Mounting device (2500) is fastened in one embodiment by mounting screw (2501).

A mounting extender (2502) may or may not be utilized with mount (2500) and is a standoff to allow for use with a number of accessories such as placement of tools, monitoring systems, positional systems, or used as fiducial markers and the like. It also can be used as a guide for placement of devices, including but not limited to, catheters, biopsy needles, stents, endoscopes, ultrasonic probes, DBS (Deep Brian Stimulation) leads or other intraoperative devices, placement of temporary or permanent monitoring, measuring or diagnostic devices in, therethrough, or around bone or other anatomy. Guidance line (2503) is a trajectory made available by mounting extender (2502) for use with various accessories. Mounting extender (2502) may articulate to align with and or provide various angles of guidance line (2503). For example the extender (2502) may rotate anywhere from 0-180 degrees to change such as placement of tools, monitoring systems, positional systems, or used as fiducial markers or use with accessories such as but not limited to catheters, biopsy needles, stents, endoscopes, ultrasonic probes, DBS (Deep Brian Stimulation) leads or other intraoperative devices, placement of temporary or permanent monitoring, measuring or diagnostic devices in, therethrough, or around bone or other anatomy.

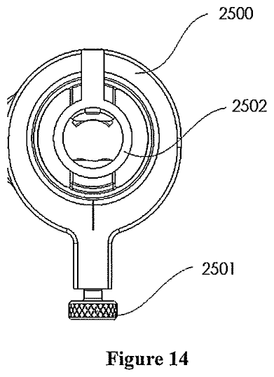

FIG. 13 depicts one embodiment showing the mounting device (2500) without the extender (2502) and intended to guide other devices, such as but not limited to, catheters, biopsy needles, stents, endoscopes, ultrasonic probes, DBS leads or other intraoperative devices and the like that are in, therethrough, or around bone or other anatomy. Again any of the devices described herein or that utilize the principles of the invention may be used with any mounting system and/or accessories.

FIG. 14 depicts a top view of one embodiment shown in FIG. 12 for placement of temporary or permanent monitoring, measuring or diagnostic devices in, therethrough, or around bone or other anatomy. It is understood that although the present invention provides for intermittent or temporary fixation to a bone structure that the device may also be used for permanent fixation such as but not limited to the purposes of monitoring, diagnostic procedures, and the like. Shown in FIG. 14 is mounting extender (2502) attached to mounting fixture or retaining ring (2500). The mounting extender (2502) shown provided for a circular or concentric holder, but such holder is not so limited to use with the present bone fixation device. Also shown is mounting screw (2501) that when tightened attaches mount (2500) onto the present bone fixation device.

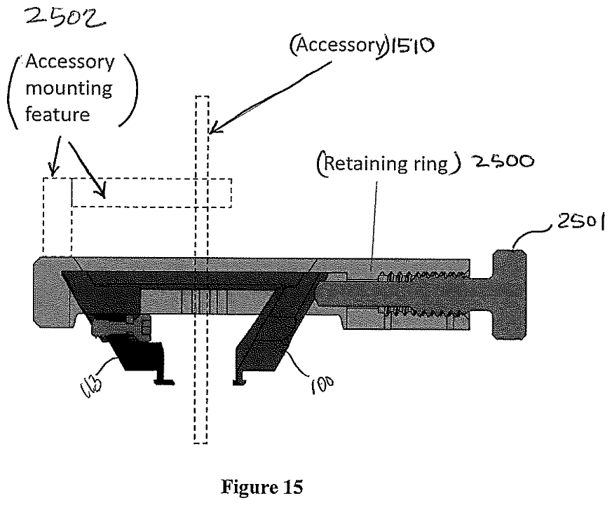

FIG. 15 illustrates a cross sectional view of one embodiment of the present invention with a mounting feature (2500) and an accessory (1510) and mounting extender feature (2502). Again the accessory (1510) may be any item including but not limited to catheters, biopsy needles, stents, endoscopes, ultrasonic probes, DBS (Deep Brian Stimulation) leads or other intraoperative devices, placement of temporary or permanent monitoring, measuring or diagnostic devices in, therethrough, or around bone or other anatomy. The accessory (1510) is not limited to just one accessory and may include several accessories attached to or used in connection with the present bone fixation device.

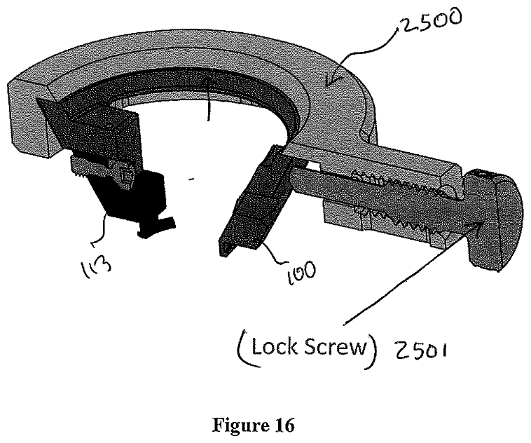

FIG. 16 is a cross sectional view of the mounting feature shown in FIG. 15 without the mounting extender feature (2502) and accessory (1510). As shown mounting screw (2501) or locking screw contacts the present invention bone fixation device to lock the mounting device (2500) thereto. Again no particular mounting mechanism is required for the present invention as the present bone fixation device may be utilized with various mounting devices and configurations and various accessories.

Any headings and sub-headings utilized in this description are not meant to limit the embodiments described thereunder. Features of various embodiments described herein may be utilized with other embodiments even if not described under a specific heading for that embodiment.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

What is claimed is:

1. A bone fixation device, comprising:
a base disposed above a bone structure having a bone burr hole or a bone hole with an inner wall; the base having a geometrical shape or a ring shape;
a plurality of protrusions or a plurality of feet connected to the base and inserted inside the bone burr;
the feet are in contact with and apply pressure to the inner wall of the bone hole regardless of a diameter of the bone burr for holding the bone fixation device onto a bone structure having a bone surface;
the bone fixation device is secured flat onto the bone structure without screws or other fixation accessories into the bone surface of the bone structure; and
wherein the feet is only in contact with the inner wall of the bone hole for holding of the bone fixation device.

2. The device of claim 1, wherein the number of feet are between 2 to 4, and the feet are of a design that penetrate bone without requiring rotation of a portion of the feet in contact with bone.

3. The device of claim 2, wherein the bone hole has a 14 mm, 11 mm, or 8 mm diameter.

4. The device of claim 1, further including a spring action member or hinge having a material deflection without deformation to apply pressure to the inner wall of burr hole.

5. The device of claim 1 further having a removable or temporary feature wherein the device is not intended for permanent fixation; and removal of the device does not cause clinically relevant damage to the bone.

6. The device of claim 1, wherein the feet further includes a rough feature for contact into the inner wall of the burr hole.

7. The device of claim 6, further includes a protrusion or a sharp for biting into or engaging the sidewall of the burr hole as the device is expanded.

8. The device of claim 6, wherein the teeth has a triangular profile or a surface selected from the group consisting of a knurl, a roughened surface, a textured surface, a raised surface, a tapered protrusion, a knife edge, and any combination thereof, and the profile depends on an expected loading of the fixation device, wherein the expected loading includes pullout, rotation, and torque.

9. The device of claim 8, wherein an arc length of the profile are within the burr hole and used for at least one accessory to go into or through the burr hole.

10. The device of claim 6, wherein the teeth are multiple rows of teeth and dependent on a clinical need.

11. The device of claim 1, the fixation device further defines a clearance through a center of the fixation device for allowing access to and/or through the burr hole.

12. The device of claim 11, wherein the clearance further includes a lateral access defined by the fixation device, and wherein the lateral access provides a space available from a side of the fixation device to visualize and access at least one accessory.

13. The device of claim 11, wherein the at least one accessory is selected from the group consisting of an implant, an instrument, a catheter, a needle, a biopsy needle, a stent, an endoscope, an ultrasonic probe, a DBS lead, an intraoperative device, a sensor, a registration, a fiduciary marker, a diagnostic device, a guide, a surgical device, equipment, and any combination thereof.

14. The device of claim 1, further includes a flat surface that does not deform or deflect, and wherein the flat surface is a reference surface for interface with other components.

15. The device claim 1, wherein the fixation device is seated flat regardless of the curvature of the bone structure.

16. The device of claim 1, further includes an expandable hinge for expansion of the fixation device; and expansion of the fixation device is achieved without an external instrument.

17. The device of claim 16, wherein the hinge is a different material than the fixation device to accommodate strain during use of the fixation device.

18. The device of claim 1, wherein the teeth provides for vertical axis tensioning when the teeth are expanded radially and/or tangentially relative to surface of the bone structure.

19. The device of claim 1, further including a bone retractor to apply pressure to the inner wall of the burr hole for mounting of accessories.

20. The device of claim 1, further including a mount attached to an external surface of the fixation device, the mount for holding accessories thereon.
